(19) United States
(12) Patent Application Publication
(10) Pub. No.: US 2004/0024322 A1
(43) Pub. Date: Feb. 5, 2004

(54) Pulsating pressure chamber and method for enhanced blood flow

(76) Inventor: Carl A. Caspers, Avon, MN (US)

Correspondence Address: Lynn C. Cameron, FAEGRE & BENSON LLP, 2200 Wells Fargo Center, 90 South Seventh Street, Minneapolis, MN, 55402-3901, US

(21) Appl. No.: 10/444,745

(22) Filed: May 23, 2003

Related U.S. Application Data
(60) Provisional application No. 60/383,159, filed on May 23, 2002.

(51) Int. Cl. A61B 5/02

(52) U.S. Cl. 600/481

(57) ABSTRACT

An apparatus and method for providing positive and negative pressure to a portion of a body for the purpose of improving blood flow in that portion of the body. The apparatus including a pressure chamber configured to generally conform to and surround a portion of the body, with the chamber adapted to receive the portion of the body within an opening in the chamber. The chamber also including a flexible liner adapted to surround the portion. The liner is configured to extend beyond the chamber when the portion covered by the liner is placed within the chamber with the chamber sealing to the liner. Additionally including a pressure control device having sources of positive and negative pressure. The device being operably connected to the chamber to provide positive and negative pressure to the portion within the chamber in a predetermined, controlled manner. A pressure patch is also described.

This application claims the benefit of U.S. Provisional Application, Serial No. 60/383,159, filed on May 23, 2002 and entitled PULSATING BASIC PRESSURE CHAMBER FOR ENHANCED BLOOD FLOW.

### FIELD OF THE INVENTION

This invention relates to pressure chambers used to enhance blood flow in body parts.

### BACKGROUND OF THE INVENTION

The use of positive pressure, negative pressure, and alternating positive and negative pressure on a body part to promote wound healing and to enhance blood flow has been shown previously.

Among other things, there is a need for an apparatus to apply alternating positive and negative pressure to a body part wherein the apparatus is programmably controlled in order to control the amplitude and frequency of the changes in pressure and improve the therapeutic effect.

### SUMMARY OF THE INVENTION

The present invention is directed to an apparatus for enhancing blood flow to a body part through the use of alternating positive and negative pressure. In particular, the invention relates to such an apparatus is controlled in order to control the amplitude and/or frequency of the changes in pressure.

The present invention provides an apparatus for providing positive and negative pressure to a portion of a body for the purpose of improving blood flow in that portion of the body. In one embodiment, the apparatus includes a pressure chamber configured to generally conform to and surround a portion of the body, with the pressure chamber adapted to receive the portion of the body within an opening in the pressure chamber. The apparatus also includes a flexible liner adapted to surround the portion of the body. The liner is configured to extend beyond the pressure chamber when the portion of the body covered by the liner is placed within the pressure chamber, with the pressure chamber sealing to the liner. In addition, the apparatus has a pressure control device including a source of positive pressure and a source of negative pressure. The pressure control device is operably connected to the pressure chamber to provide positive and negative pressure to the portion of the body within the pressure chamber in a predetermined, controlled manner.

In another embodiment, the apparatus includes a pressure patch configured to generally conform to a portion of the body. The pressure patch is adapted to cover a discrete surface area of the portion of the body. The apparatus also includes a flexible liner adapted to be interposed between the surface area of the portion of the body and the pressure patch, with the flexible liner sized to cover a greater area of the body than the portion of the body under the pressure patch. The pressure patch seals to the flexible liner. The apparatus further includes a pressure control device including a source of positive pressure and a source of negative pressure. The pressure control device is operably connected to the pressure patch to provide positive and negative pressure to the portion of the body covered by the pressure patch in a predetermined, controlled manner.

The present invention also includes a method of enhancing blood flow in a portion of a body. The method includes the steps of covering a portion of the body with a liner and applying positive and negative pressure to the liner. The liner conveys the applied pressure to the portion of the body. The method further incldues controlling the applied pressure.

A principal object and advantage of the present invention is that increases blood flow to a body part.

Another principal object and advantage of the present invention is that it includes a computer that can regulate the change in amplitude and frequency in the positive and negative pressure to produce a wave of any shape.

### BRIEF DESCRIPTION OF THE SEVERAL VIEWS OF THE DRAWINGS

FIG. 1 is a schematic of the apparatus with a body part inserted.

FIG. 2 is a detailed schematic of one embodiment of the apparatus and control mechanism.

FIG. 3 is a flowchart of a control loop for the present invention.

FIGS. 4 and 5 are charts showing the variation of blood flow to a body part compared to the pressure cycle produced by the apparatus.

### DETAILED DESCRIPTION OF THE INVENTION

With reference to the attached Figures, it is to be understood that like components are labeled with like numerals throughout the several Figures. The present invention includes an apparatus for providing pressure therapy as alternating positive and negative pressure in an enclosure surrounding a portion of a body for the purpose of improving blood flow.

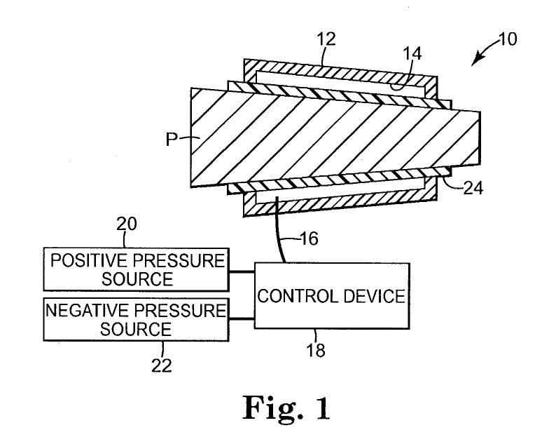

A schematic of a first embodiment of the apparatus 10 of the present invention is shown in FIG. 1. The apparatus 10 preferably comprises a sealed chamber 12 having an interior 14 with a pressure line 16 entering the interior 14. The pressure line 16 is connected to a control device 18. The control device 18 is, in turn, connected to a positive pressure source 20 and a negative pressure source 22. Although shown as separate positive and negative pressure sources 20, 22, it is to be understood that a single source capable of providing both positive and negative pressures may also be used.

In the first embodiment, a body part “P” is enclosed in a liner 24, and the body part P and liner 24 are inserted into the chamber 12. The liner 24 is preferably constructed of a flexible urethane, although other materials may also be used. The chamber 12 can be configured to generally conform to the shape of the body part P (such as a trunk, hand, arm, foot, leg, portions thereof or the residuum thereof following a partial amputation thereof), as shown in FIG. 1. That is, the chamber 12 can be anatomically shaped to conform to and surround the portion of the body part to which pressure therapy is to be applied. This configuration reduces the overall volume of the chamber as compared to prior pressure chambers usually formed as large boxes into which a body part is placed. Reduced volume of the chamber 12 results in enhanced control over the fluid volume in the body part P, thereby reducing the occurrence or degree of edema, the accumulation of fluid in a body part. Better control over fluid volume provides more variability in the time and amount of pressure therapy that may be applied to the body part, without causing or at least reducing negative side effects. That is, a smaller volume chamber, such as chamber 12, can allow for longer pressure therapy sessions or overall duration without causing or at least reducing edema or other problems for the patient. The chamber 12 can be said to provide a means for applying pressure to the portion of the body or body part P.

The liner 24 can extend over the body part P and beyond the boundaries of the chamber 12, such that the chamber 12 can seal to the liner 24 and not to the body part P directly, as shown in FIG. 1. As a result, the positive and negative pressure are applied to the liner 24 and not the skin of the patient. Use of the liner 24, such as a urethane liner, allows for equalization of the pressure being applied (both positive and negative). Certain urethane liners are known to be semi-fluid and allow for the distribution or re-distribution of force over the surface area of the urethane liner. As a result, the body part P is subjected to less trauma and thus more beneficial pressure therapy may be provided. The liner 24 can be said to provide a means for sealing and/or a means for equalizing, distributing, or redistributing pressure.

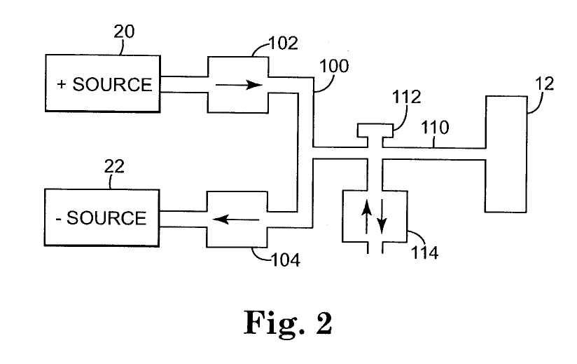

Pressure in the chamber 12 can then be controlled by means of a circuit, such as, for example, the device 18. The device 18 controls the amplitude of the positive and negative pressure and/or the frequency at which the positive and negative pressure can be oscillated. One embodiment of a representative pneumatic schematic diagram is shown in FIG. 2. In this embodiment, a pneumatic connection 100 can be coupled to the positive pressure source 20 and is controlled by a normally closed electronic solenoid 102. The pneumatic connection 100 can also be coupled to the negative pressure source 22 and is controlled by a normally closed electronic solenoid 104. The pneumatic connection 100 can be coupled to the chamber 12 via a second connection 110, in which an absolute pressure sensor 112 and a bleed to atmosphere 114 can be fluidly coupled. The absolute pressure sensor 112 can be configured to sense both positive and negative pressures. The bleed to atmosphere 114, as shown, can also be a normally closed electronic solenoid. Computer or other programmable control of the solenoids 102, 104, 114 and sensor 112 provide the desired pressure cycles within the chamber 12, as well as better optimization, increased control of the pressure chamber and pressure therapy, and feedback capability. All or portions of the components noted in this paragraph can be referred to as means for controlling pressure.

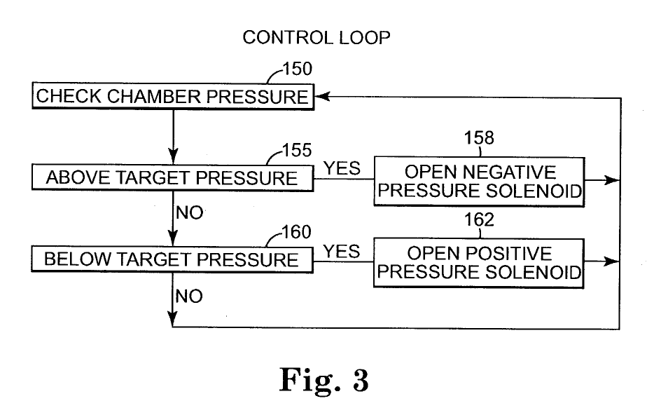

One embodiment of a representative pressure control flow chart is shown in FIG. 3. In this embodiment, the pressure of the chamber 12 can be checked in step 150. The pressure can then be compared to a desired targeted value and evaluated as to whether the sensed pressure is above the targeted value, in step 155. If so, the negative pressure solenoid 104 can be opened, in step 158. The pressure can then be checked again in step 150. If not, the sensed pressure can be evaluated as to whether it is below the targeted value, in step 160. If so, the positive pressure solenoid 102 can be opened, in step 162. The pressure can then be checked again in step 150. If not, the pressure can be checked again in step 150. This loop can be repeated as needed during the pressure therapy session.

Preferably, the positive and negative pressure is applied to the body part P in a wave cycle (preferably a sine wave). This wave could be altered or possibly synchronized to the rhythm of the heart to assist in blood flow. In one embodiment, the control device 18 can be coupled to a heart rate monitor or other sensor allowing for feedback to the control device 18 in order to synchronize the pressure wave cycle to the rhythm of the heart of the patient or the blood flow in the particular body part being treated.

As the positive pressure is applied, blood can be evacuated from the body part P in the area inside the chamber. When the negative pressure is applied, blood is pulled into the body part P in the area in the chamber. Since blood can flow only in one direction in a blood vessel of the human body, this is intended to provide a fresh supply of blood to the body part P in the area inside the chamber.

In a second embodiment, the apparatus may comprise a bandage or patch that makes a sealed contact with an area of the skin, rather than a chamber into which a body part is inserted. As with the first embodiment, a liner portion is provided as part of the bandage or patch over the skin with the pressure portion applied to the liner portion, instead of directly to the skin.

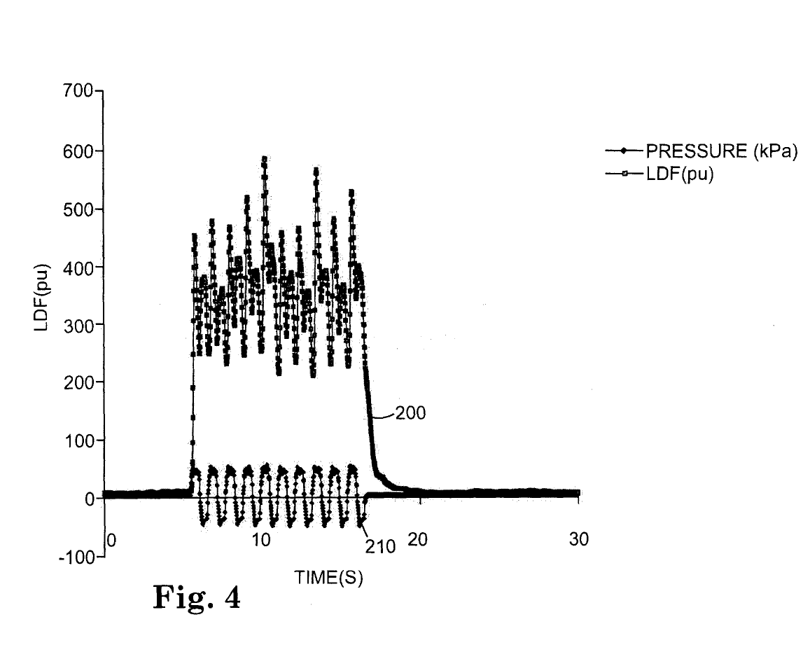

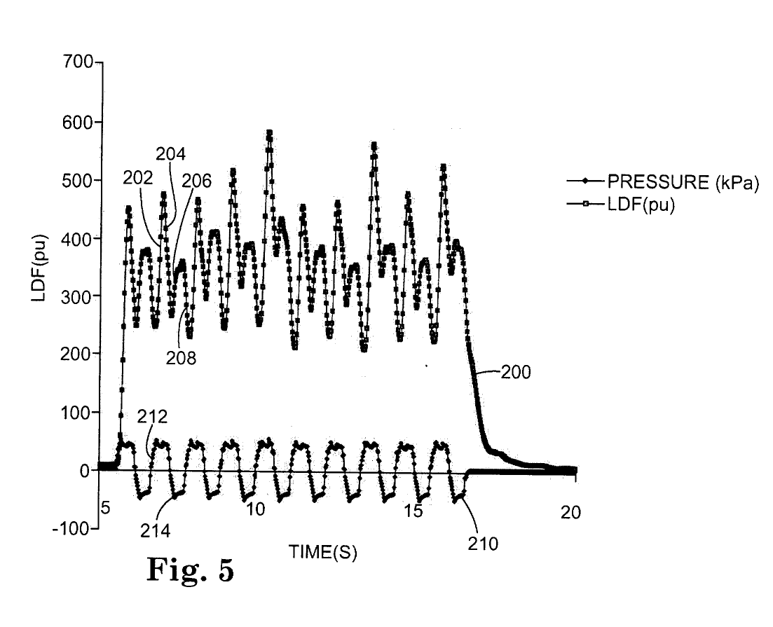

FIGS. 4 and 5 show the relationship between measured blood flow 200 in the body part and the pressure cycle 210 applied to the body part. As can be seen in the graphs, and most clearly in FIG. 5, as a positive pressure 212 can be applied, blood flow increases 202 until the positive pressure constricts the vessels or significantly reduces the fluid volume in the body part, thus causing blood flow to drop 204. The subsequent application of a negative pressure 214 results in another increase in blood flow 206, but only to a limit at which time the blood flow again drops off 208. This cycle is repeated as the positive and negative pressures are repeated. Overall, however, blood flow is generally increased during the pressure therapy. Most preferably, the range of pressures that the apparatus may employ is from 30 to 760 mm. Hg (0.6 to 15 psi) positive pressure and from −30 to −640 mm. Hg. (−0.6 to −12 psi) negative pressure. Cycle frequency may vary, but it can be about 1 second/per cycle, as shown in FIGS. 4 and 5 or it can be optimized according to avoid reaching or closely approaching constrictive limits causes by a duration of positive or negative pressure.

The present invention may be embodied in other specific forms without departing from the spirit or essential attributes thereof, and it is therefore desired that the present embodiment be considered in all respects as illustrative and not restrictive.

What is claimed is:

1. An apparatus for providing positive and negative pressure to a portion of a body for the purpose of improving blood flow in that portion of the body, the apparatus comprising:
a pressure chamber adapted to receive the portion of the body through an opening in the pressure chamber;
a liner adapted to surround the portion of the body, the liner configured to extend beyond the pressure chamber when the portion of the body covered by the liner is placed within the pressure chamber with the pressure chamber sealing to the liner; and
a pressure control device including a source of positive pressure and a source of negative pressure, the pressure control device operably connected to the pressure chamber to provide positive and negative pressure to the portion of the body within the pressure chamber in a controlled manner.

2. The apparatus of claim 1, wherein the pressure chamber is configured to generally conform to and surround a portion of the body, wherein the liner is flexible, and wherein the controlled manner is a predetermined manner.

3. The apparatus of claim 1, wherein the pressure chamber has a chamber volume and the portion of the body has a body volume, and wherein the chamber volume is configured to correspond to the body volume so as to reduce the occurrence or degree of edema in the portion of the body due to the application of pressure by the pressure chamber.

4. The apparatus of claim 1, wherein the liner distributes the applied pressure over a surface of the portion of the body.

5. The apparatus of claim 4, wherein the liner comprises urethane.

6. The apparatus of claim 1, wherein the source of positive pressure and the source of negative pressure are separate sources.

7. The apparatus of claim 1, wherein the positive and negative pressures provided within the pressure chamber are oscillated at a predetermined amplitude and frequency by the pressure control device for a desired time period.

8. The apparatus of claim 7, wherein the oscillated positive and negative pressures are provided in a wave cycle.

9. The apparatus of claim 8, wherein the pressure control device synchronizes the pressure wave cycle to a predetermined input.

10. The apparatus of claim 9, wherein the pressure control device further comprises a heart rate sensor couplable to the heart of the body, and wherein the pressure control device synchronizes the pressure wave cycle to the sensed heart rate of the user.

11. The apparatus of claim 1, wherein the pressure control device comprises a programmable device.

12. The apparatus of claim 11, wherein the programmable device comprises one or more of a computer, a microprocessor and a programmable controller.

13. An apparatus for providing positive and negative pressure to a portion of a body for the purpose of improving blood flow in that portion of the body, the apparatus comprising:
means for applying pressure to a portion of the body;
means for distributing applied pressure over the portion of the body; and
means for controlling pressure being applied to the portion of the body.

14. The apparatus of claim 13, wherein the means for applying pressure comprises a pressure chamber adapted to receive the portion of the body through an opening in the pressure chamber.

15. The apparatus of claim 13, wherein the means for distributing pressure further comprises a means for sealing with respect to the means for applying pressure.

16. The apparatus of claim 13, wherein the means for distributing pressure comprises a liner adapted to surround the portion of the body, the liner configured to seal to means for applying pressure.

17. The apparatus of claim 13, wherein the means for controlling pressure comprises a pressure control device including a source of positive pressure and a source of negative pressure, the pressure control device operably connected to the means for applying pressure to provide positive and negative pressure to the portion of the body in a controlled manner.

18. The apparatus of claim 13 wherein the means for controlling pressure comprises a programmable device.

19. The apparatus of claim 18, wherein the programmable device comprises one or more of a computer, a microprocessor and a programmable controller.

20. An apparatus for providing positive and negative pressure to a portion of a body for the purpose of improving blood flow in that portion of the body, the apparatus comprising:
a pressure patch configured to generally conform to a portion of the body, the pressure patch adapted to cover a discrete surface area of the portion of the body;
a liner adapted to be interposed between the surface area of the portion of the body and the pressure patch with the flexible liner sized to cover a greater area of the body than the portion of the body under the pressure patch, the pressure patch sealing to the flexible liner; and
a pressure control device including a source of positive pressure and a source of negative pressure, the pressure control device operably connected to the pressure patch to provide positive and negative pressure to the portion of the body covered by the pressure patch in a predetermined, controlled manner.

21. The apparatus of claim 20, wherein the liner surrounds the portion of the body.

22. The apparatus of claim 20, wherein liner is flexible.

23. The apparatus of claim 20, wherein the liner distributes the applied pressure over a surface of the portion of the body.

24. The apparatus of claim 20, wherein the liner comprises urethane.

25. The apparatus of claim 20, wherein the source of positive pressure and the source of negative pressure are separate sources.

26. The apparatus of claim 20, wherein the positive and negative pressures provided within the pressure patch are oscillated at a predetermined amplitude and frequency by the pressure control device for a desired time period.

27. The apparatus of claim 26, wherein the oscillated positive and negative pressures are provided in a wave cycle.

28. The apparatus of claim 27, wherein the pressure control device synchronizes the pressure wave cycle to a predetermined input.

29. The apparatus of claim 28, wherein the pressure control device further comprises a heart rate sensor couplable to the heart of the body, and wherein the pressure control device synchronizes the pressure wave cycle to the sensed heart rate of the user.

30. The apparatus of claim 20, wherein the pressure control device comprises a programmable device.

31. The apparatus of claim 30, wherein the programmable device comprises one or more of a computer, a microprocessor and a programmable controller.

32. A method of enhancing blood flow in a portion of a body, the method comprising the steps of:
covering a portion of the body with a liner;
applying positive and negative pressure to the liner, the liner conveying the applied pressure to the portion of the body; and
controlling the applied pressure.

33. The method of claim 32, wherein the step of controlling the pressure comprises oscillating the positive and negative pressures at desired amplitudes and frequencies (it could be based on a measurement of the tissue rather than a specific duration).

34. The method of claim 32, wherein the step of covering comprises surrounding the portion of the body with the liner.

35. The method of claim 32, wherein the liner comprises flexible urethane.

36. The method of claim 32, wherein the step of applying pressure comprises inserting the portion of the body covered by the liner into a pressure chamber adapted to provide the positive and negative pressures.

37. The method of claim 36, wherein the step of applying pressure further comprises sealing the pressure chamber to the liner covering the portion of the body.

38. The method of claim 32, further comprising the step of sensing blood flow within the body and controlling the applied pressure based on the sensed blood flow.

39. The method of claim 38, wherein the step of sensing comprises sensing a heart rate of the body, and wherein the step of controlling the applied pressure comprises synchronizing the applied pressure to the heart rate of the body.
